(19) United States
(12) Patent Application Publication
(10) Pub. No.: US 2012/0149983 A1
(43) Pub. Date: Jun. 14, 2012

(54) ENDOSCOPIC CANNULA AND METHODS OF USING THE SAME

(75) Inventor: Albert K. Chin, Palo Alto, CA (US)

(73) Assignee: Pavilion Medical Innovations

(21) Appl. No.: 13/237,537

(22) Filed: Sep. 20, 2011

Related U.S. Application Data
(60) Provisional application No. 61/384,525, filed on Sep. 20, 2010.

(51) Int. Cl. A61B 1/01

(52) U.S. Cl. 600/114

(57) ABSTRACT

The present disclosure provides methods and systems for endoscopic visualization inside the human body and performing clinical procedures under endoscopic guidance. In one embodiment, an endoscopic cannula of the present disclosure may include an elongated body having a channel along which an endoscope can be advanced. The cannula further includes a tip about a distal end of the elongated body, which can be inflated from a substantially firm collapsed state to an expanded state. The cannula also includes a tube situated within the channel and attached at its distal end to a distal region of the tip to permit forward advancement of the tube along with the tip when the tip is expanded. The tube in combination with the channel form a working conduit for passing surgical instruments beyond the tip.

### RELATED APPLICATIONS

This application claims priority to and benefits of U.S. Provisional Application No. 61/384,525, filed Sep. 20, 2010, which is hereby incorporated herein by reference in its entirety for the teachings therein.

### TECHNICAL FIELD

The presently disclosed embodiments relate to endoscopic cannulas and methods of using such devices, and more particularly to endoscopic cannulas having an inflatable tip for use in connection with dissection and viewing anatomical structures.

### BACKGROUND

There currently exist dissection instruments that inflate a large balloon, approximately 1 liter size, between the layers of tissue to separate the tissues and create a working space between the layers of tissue. Some dissection balloons incorporate a viewing lens to assist with endoscopic viewing of surrounding tissues during advancement of the uninflated balloon through layers of tissue. In these devices, the viewing lens is typically able to visualize only directly in front of the lens, with limited guidance capability.

To address these issues, devices were developed incorporating a transparent conical tip to perform tissue dissection under endoscopic guidance. The transparent conical tip may allow visualization of surrounding tissue in contact with the long axis of the transparent tip. However, while the relatively small size and shape of conical tips allows cannulas with such tips to be inserted through a small incision, the area of visualization that can be provided by such conical tips is limited. Moreover, such tips are too small to create sufficient surgical space for performing clinical procedures in larger body cavities, such as an abdominal cavity.

Accordingly, there is still a need in the art for endoscopic devices that provide a sufficiently large area of visualization and allow clinical procedures to be performed under endoscopic guidance, while maintaining a small profile during their delivery to a site of interest.

### BRIEF DESCRIPTION OF DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 illustrates the difference in field of view for an endoscope through tips of various shapes.

FIGS. 2A-2D illustrate an embodiment of an endoscopic cannula of the present disclosure.

FIGS. 3A-3B illustrate another embodiment of an endoscopic cannula of the present disclosure.

FIGS. 4A-4B illustrate an embodiment of a system for endoscopic repair of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### SUMMARY OF THE INVENTION

One aspect of the present disclosure provides an endoscopic cannula that includes an elongated body having a channel along which an endoscope can be advanced. The cannula further includes a tip about a distal end of the elongated body, the tip is inflatable from a substantially firm collapsed state where the tip can aid in the advancement of the body to a site of interest, to an expanded state where the tip can permit an increased area of visualization of surrounding area by the endoscope. In addition, the cannula includes a tube situated within the channel and attached at its distal end to a distal region of the tip to permit forward advancement of the tube along with the tip when the tip is expanded, the tube defining a pathway in fluid communication with the channel to form a working conduit along which one or more surgical instruments can be advanced beyond the tip.

Another aspect of the present disclosure provides a system for endoscopic viewing that includes an endoscope and an endoscopic cannula for advancing the endoscope to a site of interest. The endoscopic cannula may include an elongated body having a channel along which the endoscope can be advanced, an inflatable tip about a distal end of the elongated body to aid in the advancement of the elongated body to a site of interest and a tube situated within the channel and attached at its distal end to a distal region of the tip to permit advancement of one or more surgical instruments beyond the tip. In addition, the system for endoscopic viewing may include a positive pressure source in fluid communication with the inner cavity of the tip for inflating the tip from the collapsed state.

Yet another aspect of the presents disclosure provides a method for endoscopically viewing body structures at a site of interest. To perform the method, first an elongated body having an inflatable tip about a distal end of the elongated body and having a tube extending from the elongated body into the tip can be navigated to the site of interest. Once at the site of interest, the tip can be inflated to an expanded state with a rounded profile such that the tube is advanced forward along with the expanded tip. Subsequently, an endoscope may be directed along the elongated body into the expanded tip to view body structures at the site of interest. In an embodiment, navigating of the elongated body to the site of interest may also be performed under endoscopic guidance.

### DESCRIPTION OF SPECIFIC EMBODIMENTS

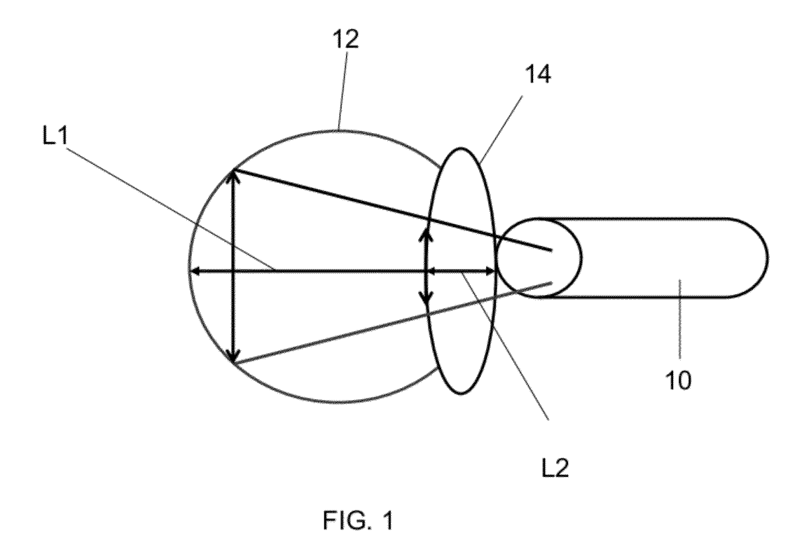

The present disclosure provides devices, methods and systems for endoscopic visualization inside the human body and performing clinical procedures under endoscopic guidance. An endoscope can be placed inside a tip of an endoscopic cannula to visualize anatomic structures in contact with the surface of the tip. In an embodiment, a relatively large diameter tip may provide an increased surface area of contact with tissue, and consequently, a larger area of visualization. Moreover, limiting the length of a balloon may narrow the field of visualization. In particular, FIG. 1 shows the difference in field of view for an endoscope 10 through a spherical balloon 12 having length L1 versus an elliptical balloon 14 having a shorter length L2.

At the same time, a smaller diameter device may be inserted into the body of a patient through a smaller incision. Moreover, a small diameter, tapered tip on the distal end of a cannula may serve as a dissector to aid in the advancement of the cannula through the body to reach target tissue or organs. Once the cannula is delivered to a site of interest, to enable a surgeon to perform procedures on tissue with endoscopic guidance, an endoscopic cannula disclosure may also be provided with a working conduit through the tip for passing surgical instruments or materials beyond the tip. In an embodiment, the working conduit may be configured to extend through both a smaller diameter and a larger diameter tip. The present disclosure provides an endoscopic cannula that combines all these features as well as systems and methods utilizing such endoscopic cannula.

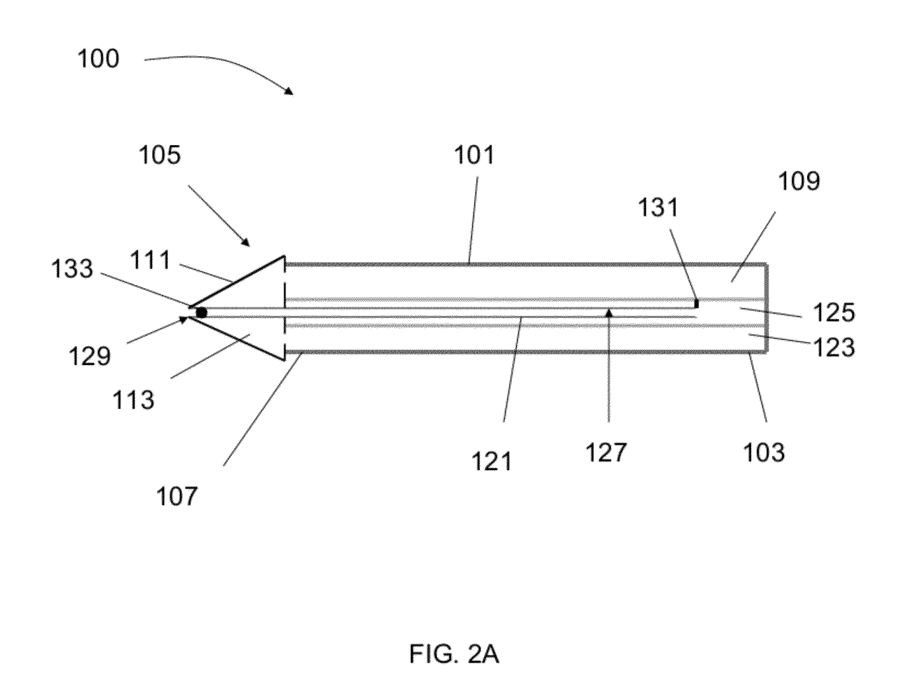

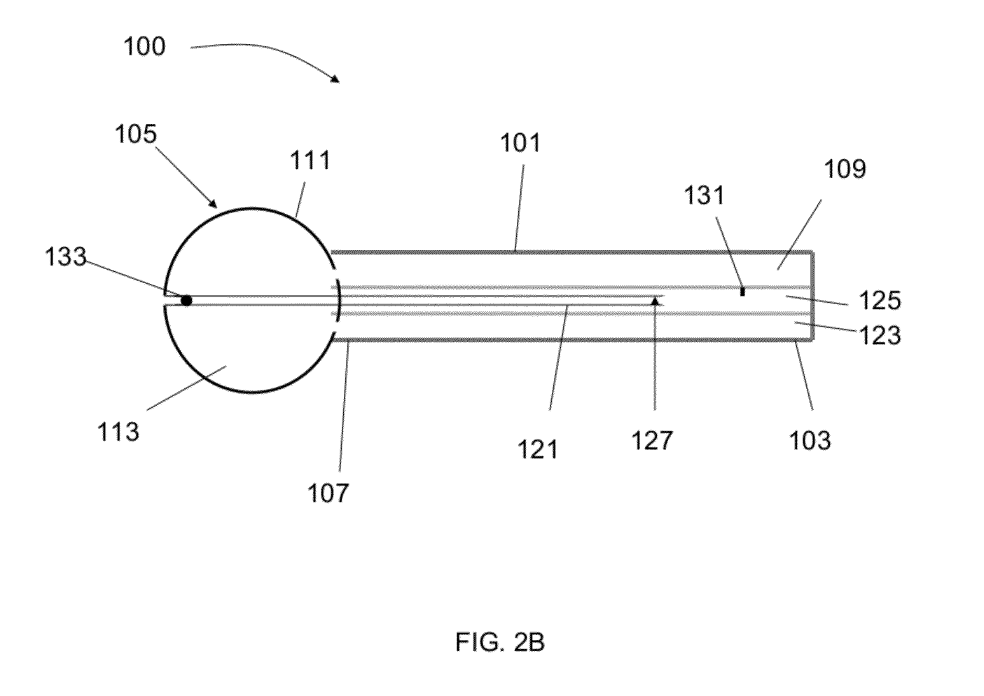

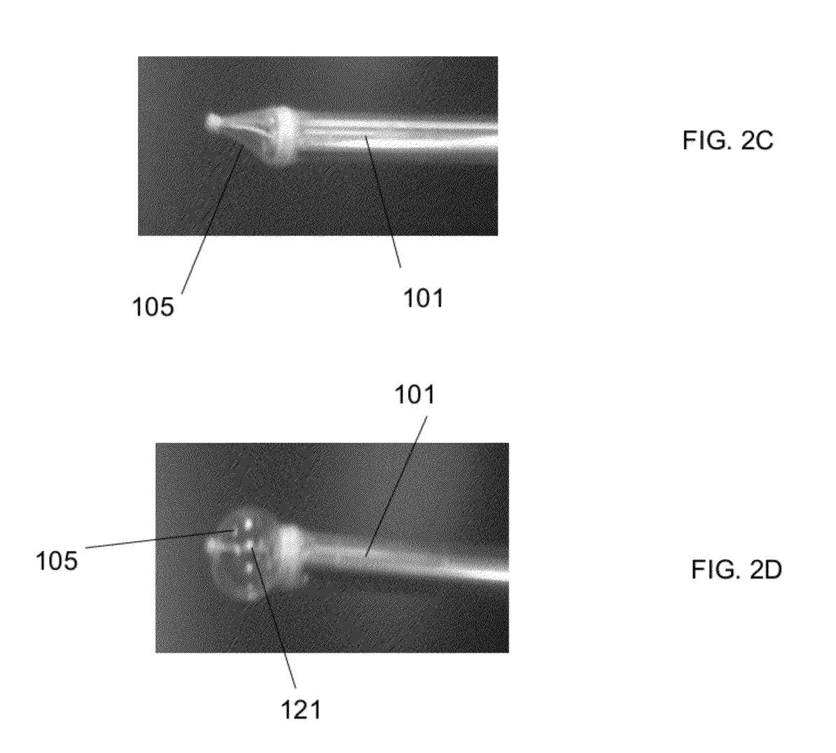

FIG. 2A and FIG. 2B illustrate an embodiment of an endoscopic cannula 100 for visualization inside the human body. In an embodiment, the endoscopic cannula 100 includes an elongated body 101 having a channel 109 extending between a proximal end 103 and a distal end 107 for passing an endoscope or other surgical instruments or materials to a site of interest. The elongated body 101 may be made of a metal or plastic biocompatible material. To aid in navigating the elongated body 101 to a site of interest, the elongated body 101 may be sufficiently rigid axially along its length, while remaining sufficiently flexible radially from side to side. To provide the elongated body 101 with such characteristic, in an embodiment, the elongated body 101 may be made from a plastic material, metallic material, shape memory material, composite material or any other materials that has the desired characteristics.

The elongated body 101 may also include material that can minimize or reduce friction, as the elongated body 101 travels to a site of interest. To further minimize friction, alternatively or additionally, the elongated body 101 may be coated with a hydrophilic coating, such as, for example, polyvinylpyrrolidone, polyurethane, poly(acrylic acid), poly(methacrylic acid), poly(dimeth)acrylamide, PTFE, poly(acrylamide), polyvinybutyrol, poly(hydroxyethylmethacrylate) or combinations thereof. The elongated member 102 may also be coated with an anti-thrombogenic coating, such as heparin (or its derivatives), urokinase, or PPack (dextrophenylalanine proline arginine chloromethylketone) to prevent thrombosis or any other adverse reaction due to the introduction of the elongated member 101 into the body of a patient. Other components of the endoscopic cannula 100, as will be described below, may also be coated with a hydrophilic coating, an anti-thrombogenic coating, or both.

The cannula 100 may further include an inflatable tip 105 disposed about the distal end 107 of the elongated body 101. The tip 105 may be transparent to allow for endoscopic viewing through the tip 105. Initially, the tip 105 may be deflated to allow the endoscopic cannula 100 to be inserted into a patient through a small incision. When in the deflated state, the tip may form a rigid, tapered profile to perform dissection as the endoscopic cannula 100 is navigated along body structures through a small access incision. The tip 105 is inflated at a site of interest to a larger diameter to increase surface area of contact of the tip with tissue such that an endoscope placed inside the tip 105 is provided with a larger area of visualization. In an embodiment, the tip 105 may be an integral part of the elongated body 101. In another embodiment, the tip 105 may be removably attached to the elongated body 101.

The tip 105 may include a wall 111 that defines an inner chamber 113. The tip 105 may be expandable from a collapsed state, as shown in FIG. 2A, to an expanded state, as shown in FIG. 2B, by injecting one or more medical grade fluids, into the inner chamber 113 of the tip. The tip 105 can be returned to the collapsed state by removing the one or more fluids from the inner chamber 113. Suitable medical grade fluids include, but are not limited to, air, nitrogen, carbon dioxide, water, saline solution, buffer solution or similar gasses or liquids. To be able to move the one or more medical grade fluids to or from the inner chamber 113 of the tip 105, the inner chamber 113 is in fluid communication with the channel 109 of the elongated body 101. A syringe or a similar positive pressure instrument can be attached at a proximal end of the channel 109, by means of, for example, Luer type fitting, to move fluids in and out of the inner chamber 113.

The tip 105 may be made from a transparent material to permit endoscopic viewing through the tip. In an embodiment, the tip 105 may be made of a resilient and puncture resistant elastomeric material. Suitable materials for manufacturing the tip 105 include, but are not limited to, urethane, polyethylene terephthalate (PET), nylon elastomer and other similar polymers.

As shown in FIG. 2A, the tip 105 may be designed to maintain a tapered profile when in the collapsed state. Examples of suitable tapered profiles include, but are not limited to, conical, beveled or similarly angled profiles. The tapered profile may assist in the dissection of tissue layers as the endoscopic cannula 100 is navigated to a site of interest. The tapered profile may also provide visualization without visual or light distortion because the relatively small size and the tapered profile of the tip 105 in the collapsed state may minimize or eliminate the reflection of the light source from the endoscope on the inner surface of the tip 105. That is, an endoscope may be inserted into the inner chamber 113 of the tip 105 through the channel 109 as the endoscopic cannula 100 is navigated to a site of interest to visualize the progress of the endoscopic cannula 100. In an embodiment, the tip 105 may be substantially firm when in the collapsed state, such that the endoscopic cannula 100 can be negotiated along body structures to a site of interest. It should be noted that, in some embodiments, the tip 105 can be somewhat flexible as long as the tip 105 does not buckle or get deflected as the endoscopic cannula 100 is advanced to a site of interest. In an embodiment, the tip 105 may be blunt to avoid injuring surrounding tissues as the endoscopic cannula 100 is navigated to a site of interest. On the other hand, to aid in tissue dissection, the tip 105 may be substantially sharp.

Once the endoscopic cannula 100 is placed at a site of interest, the tip 105 may be moved from the collapsed state to the expanded state to assume a rounded profile, as shown in FIG. 2B. The term “rounded profile” as used herein means that the tip when inflated has a smooth surface, rather than angular surface. Exemplary rounded profiles include, but are not limited to, a spherical, circular or elliptical shape. The tip 105 may be provided with a shape and/or size such that, when the tip 105 is inflated, the tip 105 provides a desired area of visualization. In general, larger surface area of contact of the tip with anatomical structures results in larger area of visualization. Moreover, the area of visualization may be increased by increasing the length of the tip 105 in the expanded state, i.e., the distance between distal edge 129 of the tip 105 and the distal end of the elongated body 101, as shown in FIG. 1. As the tip 105 is inflated, the tip 105 may further dissect tissue in proximity to the site of interest in order to generate a surgical space at the site of interest. Accordingly, in an embodiment, the tip 105 may be provided with the size and shape to generate a sufficient surgical space at the site of interest. FIG. 2C and FIG. 2D illustrate an embodiment of the endoscopic cannula 100 with the tip 105 in the collapsed position (FIG. 2C) and in the expanded position (FIG. 2D).

In an embodiment, the tip 105 can be configured to assume a spherical shape when inflated. In an embodiment, the tip 105 may be pre-shaped during manufacturing to assume a spherical shape when inflated. Alternatively or additionally, the tip 105 may be provided with an internal matrix which, when expanded due to the inflation of the tip, may provide the tip 105 with a spherical shape. The shape of the tip 105 may, in an embodiment, be also controlled by the amount of inflation of the tip 105. For example, the tip 105 may have a spherical shape when inflated with a small volume of medical grade fluid, but may have an elliptical shape when inflated with a larger volume of medical grade fluid because after a certain point of inflation, the diameter of the tip may continue to increase but the length may remain the same. As noted above, this effect may be prevented by embedding a reinforcing matrix inside the tip 105 to ensure that the tip 105 remains spherical even if inflated with a relatively large volume of medical grade fluid.

In reference to FIGS. 2A-2D, the endoscopic cannula 100 may further include a tube 121 extending between a channel 109 of the elongated body 101 and a distal edge 129 of the tip 105. The tube 121 may be made from the same or different material than the elongated body 101. In an embodiment, the tube 121 may be hollow to define a pathway 127 for advancing one or more surgical instruments or materials through the tip 105. In an embodiment, a seal 133 may be disposed in the pathway 127 to ensure that the tip 105, when inflated, remains in the expanded state as surgical instruments or materials are passed through the pathway 127. The seal 133 may be made of a material capable of forming a tight seal around a surgical instrument or material passed through the pathway 127 to prevent leakage of medical grade fluid from the tip 105 when a surgical instrument or material is passed through the inflated tip 105.

The pathway 127 may be in communication with the channel 109 to form a working conduit through which surgical instruments or materials can be passed from the proximal end of the elongated body 101, through the tip 105, and distally out of the tip 105.

As shown in FIG. 2A and FIG. 2B, the channel 109 may include one or more lumens to allow passing of surgical instruments or materials therethrough. In an embodiment, the channel 109 may include an inflation lumen 123 and an instrument lumen 125. The inflation lumen 123 may be in communication with the inner chamber 113 of the tip 105 to enable the movement of the tip 105 between the collapsed state and expanded state. Moreover, an endoscope may be inserted into the inner chamber 113 of the tip 105 through the inflation lumen 123. On the other hand, the instrument lumen 125 may be in communication with the pathway 127 defined by the tube 121 to form a working conduit for passing surgical instruments or materials through the endoscopic cannula 101. In an embodiment, the inflation lumen 123 and the instrument lumen 125 may be isolated from each other. However, should it be desired, the inflation lumen 123 and the instrument lumen 125 may be in communication with each other. It should also be noted that although FIG. 2A and FIG. 2B illustrate the channel 109 with two lumens 123 and 125, the channel 109 may only include a single lumen, which would be in communication with both the inner chamber 113 of the tip 105 and the pathway 127 of the tube 121, or more than two lumens.

The distal end of the tube 121, in an embodiment, may be sealably attached to the distal edge 129 of the tip 105. However, tube 121 may be designed to be freely moveable within the channel 109 between an initial position, when the tip 105 is in the collapsed state as shown in FIG. 2A, to an advanced position, when the tip 105 is in the expanded state as shown in FIG. 2B. In this manner, forward advancement of the tube 121 to an advanced position may be permitted as the tip 101 is inflated, while the return of the tube to the initial position is permitted as the tip 105 is deflated. In other words, as the tip 105 is inflated from its collapsed state to its expanded state, because the tube 121 is attached to the distal edge 129 of the tip 105, the tip 105 may pull along the tube 121 such that the tube 121 is advanced forward in relation to the elongated body 101 from an initial position, shown in FIG. 2A, to an advanced position, shown in FIG. 2B. In an embodiment, the tube 121 may translate along the longitudinal axis of the elongated body 101. In this manner, the tube 121, in combination with the instrument lumen 125 of the elongated member 125, may provide an extendable or floating working conduit that allows the tip to be inflated in an unrestricted manner, while maintaining a conduit for delivery of surgical instruments and materials beyond the tip 105 in both the deflated state and expanded state.

In an embodiment, the tube 121 may be utilized to provide the tip 105 with a tapered profile, while the tip 105 is in the collapsed state. To that end, in an embodiment, the tube 121 may be configured such that the tube 121, when in its initial position, extends distally a predetermined distance beyond the channel 109 to provide a desired stretching to the tip 105, such that the tip 105 is provided with the tapered profile. In an embodiment, such predetermined distance may be between about 10 mm and about 20 mm, with about 15 mm being preferred. Extending the tube 121 past the channel 109 may provide the tip 105 with a tapered profile by stretching the tip 105 at a point. In addition to providing the tip 105 with a tapered profile, stretching the tip 105 may also increase wall tension in the stretched tip membrane to add rigidity to the tapered tip for enhanced dissection during the advancement of the endoscopic cannula 100 and may increases the transparency of the tip 105 for improved endoscopic visualization. To ensure that the tip 105 is sufficiently stretched, in an embodiment, a stop 131 may be placed within the channel 109 at a pre-determined distance from the distal end of the channel 109. This distance can, in an embodiment, be calculated based on the length of the tube 121 and the desired stretching to the tip 105. In this manner, the tube 121 may be prevented from sliding into the channel 109 beyond a certain point so that the tube 121 cannot act to provide sufficient amount of stretch to the tip 105. It will of course be understood that the type of taper may be changed by changing the location where the tube 121 is attached to the distal edge 129 of the tip 105. For example, attaching the tube 121 at the midpoint of the distal edge 129 may provide the tip 105 with a conical profile, while attaching the tube 121 away from the midpoint of the distal edge 129 may provide the tip 105 with a beveled profile. It should of course be understood that the tip 105 may be provided with a tapered profile by other means. For example, a tapered reinforcing net may be embedded inside the tip 105 to provide the tip with the tapered profile when the tip is collapsed.

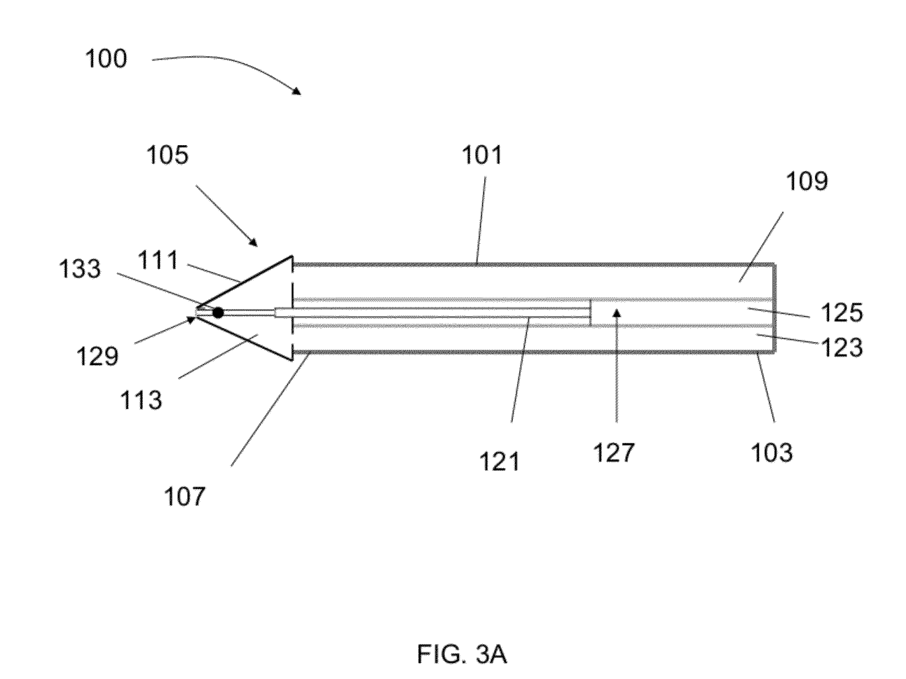

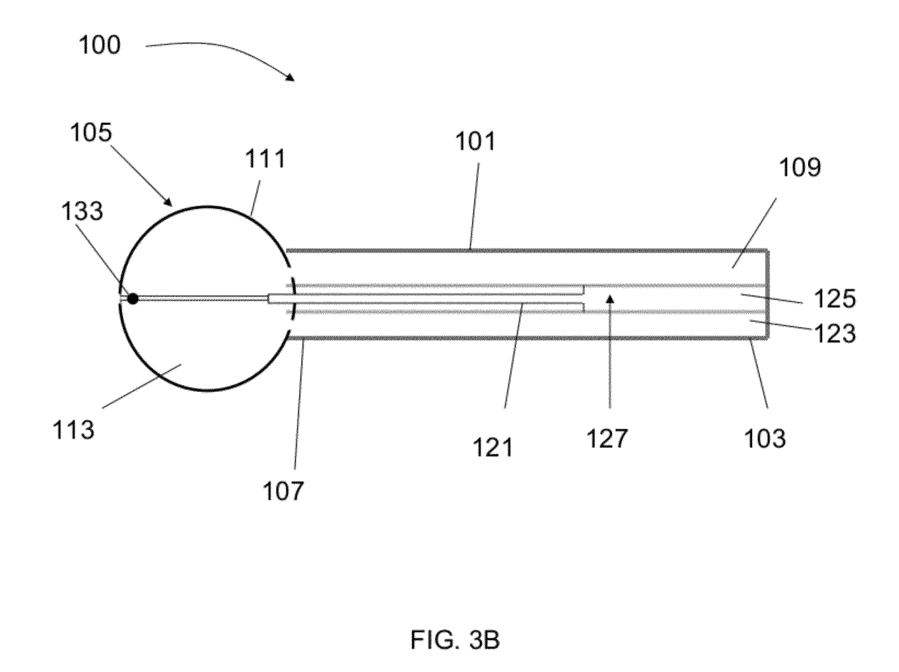

In reference to FIGS. 3A and 3B, in an embodiment, the tube 121 may be extendable or stretchable. With such design, the tube 121 can be fixed within the channel 109 of the elongated body 101 and the forward advancement of the tube 121 to an advanced position may be permitted by extending or stretching the tube 121 from an initial position as the tip 105 is inflated. By way of a non-limiting example, the tube 121 may be a telescopic tube, which can be extended from an initial position when the tip 105 is deflated, shown in FIG. 3A, to an advanced position when the tip 105 is inflated, shown in FIG. 3B, thereby providing an extendable or floating working conduit. To ensure that the tip 105 is sufficiently stretched when the tube 121 is at a initial position, in the embodiment where an extendable or stretchable tube is utilized, the minimal length of the tube 121, i.e. length when the tube is fully compressed, may be based on how far the tube 121 extends into the channel 109 of the elongated body 101 and the desired extension of the tube beyond the channel 109.

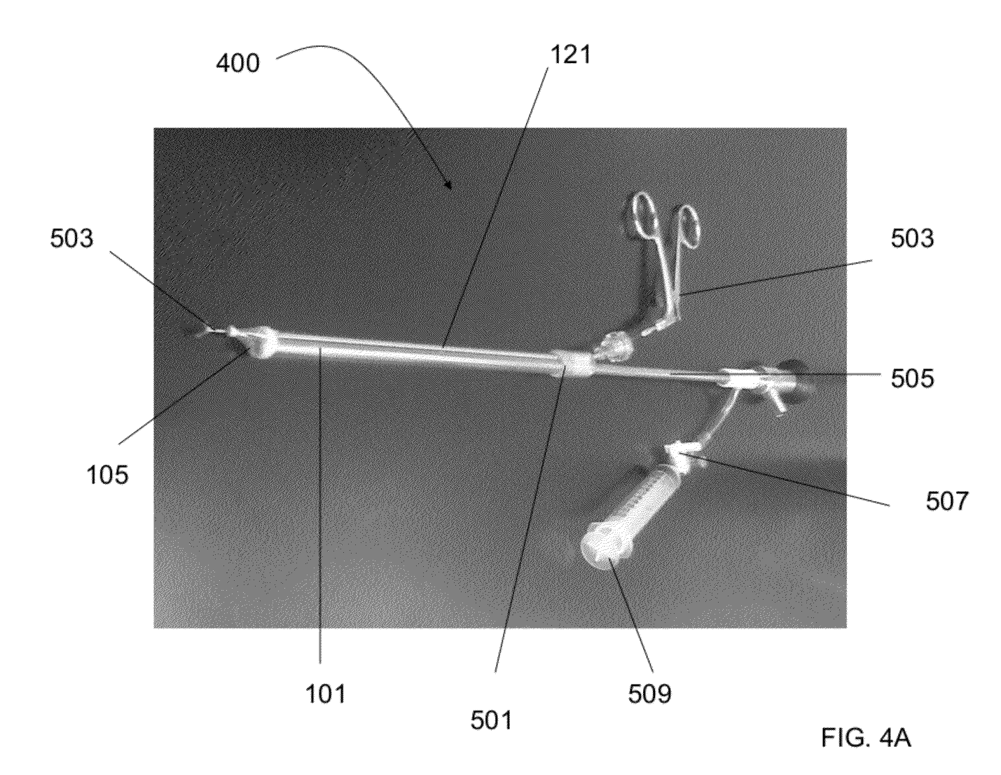

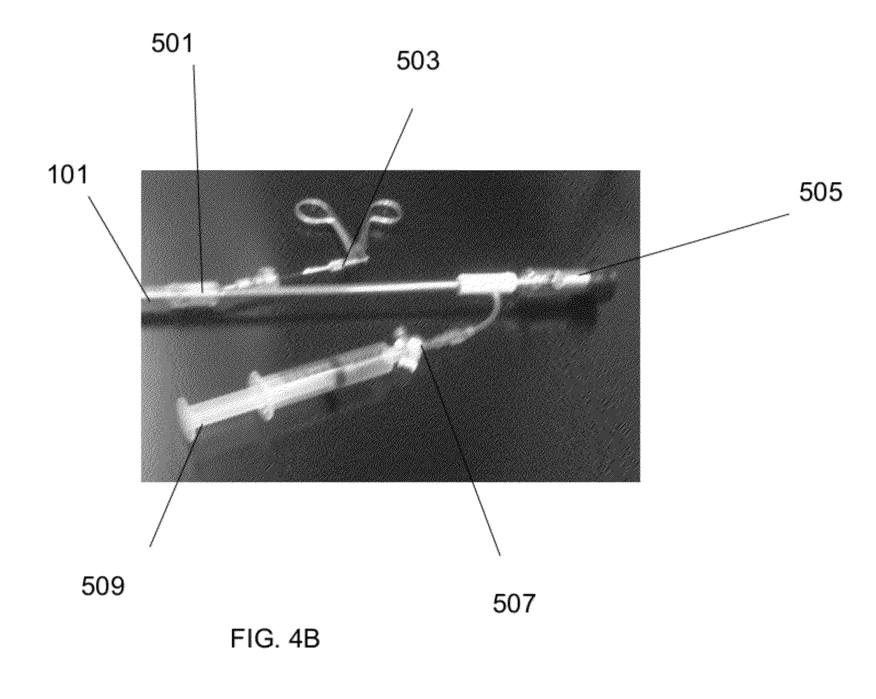

Another aspect of the present disclosure provides a system for endoscopic repair, as shown in FIG. 4. In an embodiment, a system for endoscopic repair 400 includes an endoscopic cannula 100 of the present disclosure, as shown in FIGS. 2A-3B and described in detail above. In general, the endoscopic cannula 100 may include an elongated body 101 with a tip 105 disposed about a distal end of the elongated body 101. The tip 105 may be inflatable from a collapsed state to an expanded state, and may be configured to maintain a tapered profile when in the collapsed state, while having a rounded profile when in the expanded state. The endoscopic cannula 100 may further include a tube 121 between a channel 109 of the elongated body 101 and a distal edge 129 of the tip 105. The tube 121 includes a pathway 127 in communication with the channel 109 to form a working conduit through which surgical instruments or materials can be passed from the proximal end of the elongated body 101, through the tip 105, and distally out of the tip 105.

In an embodiment, an adapter 501, such as a Tuohy-Borst adapter, may engage the proximal end 103 of the elongated body 101. Surgical instruments or materials, such as a surgical instrument 503, can be introduced into one side-arm of the adapter 501 and into the working conduit of the endoscopic cannula 100 formed by the pathway 127 in combination with a channel 109. An endoscope 505 may be introduced into another side-arm of the adapter 501 to be advanced into the inner chamber 113 of the tip 105. In addition, a syringe 509 or a similar positive pressure apparatus capable of delivering medical grade fluids may be connected to the elongated body 101 with, for example, a luer type fitting 507, to move the tip 105 between the collapsed state and expanded state.

In operation, the endoscopic cannula 100 may be inserted into a body cavity, such as an abdominal cavity, through a small percutaneous incision, and advanced to a site of interest within the body cavity. In an embodiment, a working tube may be inserted through the incision to protect the surrounding tissues by pushing the surrounding tissue out of the way to avoid cutting or tearing the surrounding tissue. The endoscopic cannula 100 may be introduced through this working tube.

As the endoscopic cannula 100 is navigated toward the site of interest, the tip 105 may be kept in the collapsed state and thus, may have a tapered profile. The tapered profile of the tip 105 may allow blunt dissection and advancement of the endoscopic cannula 100 through the body to reach target tissue or organs. Additionally, a sharp stylet may be passed through the working conduit of the endoscopic cannula 100 to aid the cannula 100 to pass through tissue and organs. The sharp tip of the stylet may protrude out of the working conduit to provide low force initial entry through tissue. As distal end of the endoscopic cannula 100 navigates within the body cavity, an endoscope may be advanced into the inner chamber 113 of the tip 105 to enable the user to view through the endoscope the inside of the body cavity and sensitive anatomic structures therein. In this manner, the user may avoid injuring sensitive anatomic structures with the endoscopic cannula 100 or the sharp stylet.

When the endoscopic cannula 100 is at the site of interest, the tip 105 may be moved from the collapsed state to the expanded state to assume a rounded profile. In an embodiment, the tip 105 is expanded to a spherical shape. As the tip 105 is moved from the collapsed state to the expanded state, the tip 105 may increase in size, thereby increasing the area that can be visualized with the endoscope inside the inner chamber 113 of the tip 105. As the tip 105 increases in size, the tip 105 also advances forward the tube 121 to maintain the working conduit through the tip. Surgical materials or instruments may be advanced through the working conduit of the endoscopic cannula 100 to perform a desired clinical procedure at the site of interest. In an embodiment, a seal may be formed around the surgical instrument to prevent leakage of medical grade fluids from the tip. Once the clinical procedure is completed, the surgical instruments may be withdrawn from the endoscopic cannula 100, tip 105 may be returned to the collapsed state and the endoscopic cannula 100 may be withdrawn from the patient.

The endoscopic cannula 100 of the present disclosure may be used for a variety of clinical applications. In an embodiment, the endoscopic cannula may be used to traverse the abdominal wall and used as a laparoscopic instrument without the need for carbon dioxide gas insufflation. The endoscopic cannula 100 may also be used to traverse through the ventricular wall into the left ventricle. The tip 105 may then be passed through the mitral valve into the left atrium, where the tip 105 can be placed at the ostia of the pulmonary veins. In an embodiment, the tip 105 may be provided with a spherical shape in the expanded state. While holding the tip 105 in position, heated fluid may be circulated through the tip to perform pulmonary vein isolation under endoscopic guidance. A balloon inflated with heated fluid has been described for endometrial ablation in the uterus (U.S. Pat. No. 4,949,718 Intrauterine cauterizing apparatus. Neuwirth and Bolduc; U.S. Pat. No. 5,105,808 Intrauterine cauterizing method. Neuwirth and Bolduc). However, this approach has not been described for pulmonary vein ostia ablation.

In one embodiment, an endoscopic cannula of the present disclosure may include an elongated body having a channel along which an endoscope can be advanced. The cannula further includes a tip about a distal end of the elongated body, which can be inflated from a substantially firm collapsed state where the tip can aid in the advancement of the body to a site of interest, to an expanded state where the tip can permit an increased area of visualization of surrounding area by the endoscope. In addition, the cannula includes a tube situated within the channel and attached at its distal end to a distal region of the tip to permit forward advancement of the tube along with the tip when the tip is expanded, the tube defining a pathway in fluid communication with the channel to form a working conduit along which one or more surgical instruments can be advanced beyond the tip.

In an embodiment, a system for endoscopic viewing of the present disclosure may includes an endoscope and an endoscopic cannula for advancing the endoscope to a site of interest. The endoscopic cannula may include an elongated body having a channel along which the endoscope can be advanced, an inflatable tip about a distal end of the elongated body to aid in the advancement of the elongated body to a site of interest and a tube situated within the channel and attached at its distal end to a distal region of the tip to permit advancement of one or more surgical instruments beyond the tip. In addition, the system for endoscopic viewing may include a positive pressure source in fluid communication with the inner cavity of the tip for inflating the tip from the collapsed state.

In an embodiment, a method for endoscopically viewing body structures at a site of interest of the present disclosure may include a step of navigating an elongated body having an inflatable tip about a distal end of the elongated body and having a tube extending from the elongated body into the tip to the site of interest. Once at the site of interest, the tip can be inflated to an expanded state with a rounded profile such that the tube is advanced forward along with the expanded tip. Subsequently, an endoscope may be directed along the elongated body into the expanded tip to view body structures at the site of interest. In an embodiment, navigating of the elongated body to the site of interest may also be performed under endoscopic guidance.

All patents, patent applications, and published references cited herein are hereby incorporated by reference in their entirety. It will be appreciated that several of the above-disclosed and other features and functions, or alternatives thereof, may be desirably combined into many other different systems or application. Various presently unforeseen or unanticipated alternatives, modifications, variations, or improvements therein may be subsequently made by those skilled in the art.

What is claimed is:

1. An endoscopic cannula, comprising:
an elongated body having a channel along which an endoscope can be advanced;
a tip about a distal end of the elongated body and being inflatable from a substantially firm collapsed state where the tip can aid in the advancement of the body to a site of interest, to an expanded state where the tip can permit an increased area of visualization of surrounding area by the endoscope; and
a tube situated within the channel and attached at its distal end to a distal region of the tip to permit forward advancement of the tube along with the tip when the tip is expanded, the tube defining a pathway in fluid communication with the channel to form a working conduit along which one or more surgical instruments can be advanced beyond the tip.

2. The endoscopic cannula of claim 1 wherein the channel permits advancement of the endoscope into the tip at the distal end of the body.

3. The endoscopic cannula of claim 1 wherein the tip has a spherical shape when inflated.

4. The endoscopic cannula of claim 1 wherein the tip is transparent to enable endoscopic viewing therethrough.

5. The endoscopic cannula of claim 1 further comprising a seal disposed in the pathway of the tube to prevent leakage of fluid from the inflated tip as one or more surgical instruments are advanced through the tip.

6. The endoscopic cannula of claim 1, wherein the tube extends from the channel into the tip to stretch the tip into the tapered profile.

7. The endoscopic cannula of claim 1 wherein the tube is slidably disposed within the channel to enable the tube to advance forward as the tip is inflated from the collapsed state to the extended state.

8. The endoscopic cannula of claim 1 wherein the tube is fixed within the channel and the forward advancement of the tube is permitted by extending the tube.

9. A system for endoscopic viewing comprising:
an endoscope;
an endoscopic cannula, comprising:
an elongated body having a channel along which the endoscope can be advanced;
an inflatable tip about a distal end of the elongated body to aid in the advancement of the elongated body to a site of interest;
a tube situated within the channel and attached at its distal end to a distal region of the tip to permit advancement of one or more surgical instruments beyond the tip;
a positive pressure source in fluid communication with the inner cavity of the tip for inflating the tip from the collapsed state.

10. The system of claim 9 wherein the tip has a spherical shape when inflated.

11. The system of claim 9 wherein the tip is configured to maintain a firm, tapered profile when deflated.

12. The system of claim 9, wherein the tube extends from the channel into the tip to stretch tip into the tapered profile.

13. The system of claim 9 further comprising a seal disposed in the pathway of the tube to prevent leakage of fluid from the inflated tip as one or more surgical instruments are advanced through the tip.

14. The system of claim 9 wherein the tube is slidably disposed within the channel to enable the tube to advance forward as the tip is inflated from the collapsed state to the extended state.

15. The system of claim 9 wherein the tube is fixed within the channel and the forward advancement of the tube is permitted by extending the tube.

16. A method for endoscopically viewing body structures at a site of interest, the method comprising:
navigating to a site of interest an elongated body having an inflatable tip about a distal end of the elongated body and having a tube extending from the elongated body into the tip;
inflating, at the site of interest, the tip to an expanded state with a rounded profile such that the tube is advanced forward along with the expanded tip; and
directing an endoscope along the elongated body into the expanded tip to view body structures at the site of interest.

17. The method of claim 16 wherein the step of navigating includes placing an endoscope into the tip to permit viewing of surrounding areas as the elongated body is navigated to the site of interest.

18. The method of claim 16 wherein the step of inflating includes expanding the tip to a spherical shape.

19. The method of claim 16 further comprising a step of advancing one or more surgical instruments into the tube and beyond the inflated tip to perform a clinical procedure.

20. The method of claim 19 further comprising a step of forming a seal around the one or more surgical instruments to prevent leakage of fluid from the tip.
